(19) United States
(12) Patent Application Publication
(10) Pub. No.: US 2004/0138592 A1
(43) Pub. Date: Jul. 15, 2004

(54) Medical indicator plaster

(76) Inventors: Reinhard Zick, Lingen/Ems (DE); Rudolf Thissen, Wassenberg (DE); Alois Cremerius, Dusseldorf (DE)

Correspondence Address: WILLIAM COLLARD, COLLARD & ROE, P.C., 1077 NORTHERN BOULEVARD, ROSLYN, NY, 11576, US

(21) Appl. No.: 10/475,465

(22) PCT Filed: Jul. 1, 2002
(86) PCT No.: PCT/DE02/02373

(30) Foreign Application Priority Data
Jul. 26, 2001 (DE) 101 36 106.8
Jul. 26, 2001 (DE) 101 36 107.6

(51) Int. Cl. A61B 5/103

(52) U.S. Cl. 600/592

(57) ABSTRACT

The invention relates to a medical indicator plaster for the use in the early recognition of diabetic foot. An economical, powerful and easy-to-use agent is produced. The agent contains a pressure-proof spherical element and/or pressure indicator film integrated into the plaster.

The invention relates to a medical indicator plaster that is applied to the sole of the foot and used in the early detection of the diabetic foot. The indicator plaster can be referred to as, or realized in the form of a measuring foil or measuring plaster. Furthermore, the invention concerns the use of the plaster as a medical diagnostic means.

The plaster is intended to be applied in the early detection of the so-called diabetic foot (briefly: DF). Among the various resultant diseases of the diabetes mellitus (briefly: DM), the diabetic foot assumes a special position. Year for year, the disease continues to cause in Germany approximately 28,000 amputations, and its consequence is not only a cost factor of the first rank for the legal health insurance, but primarily constitutes for the afflicted patent an excess measure of pain, impairments and social disadvantages as well.

In practical life, in the foreground is not only the foot as such that has to be prevented from becoming a diabetic foot (DF), but the intact foot, furthermore, represents a synonym for the preserved mobility of the diabetic. It is exactly this mobility that is important for the following reason: in the overall concept of the treatment of diabetes mellitus, it is known that the adjustment of the blood sugar (briefly: BS-adjustment) close to the standard level ranks as the highest value. This adjustment of the blood sugar (DS adjustment) close to the standard value, however, can be successfully achieved only if the possibility is maintained for the patient to positively influence his or her blood sugar level through regular muscle activity that is conducted according to the regimen. Exactly for this activity, the male or female patient needs healthy feet.

Therefore, a diabetic with the DF-syndrome is afflicted not only with local problems afflicting his or her feet, but, furthermore, is faced with a permanent problem with his or her BS-level as well. The treatment goal of a blood sugar adjustment close to the standard level is difficult to reach, if it can be reached at all, under the condition of the DF-syndrome. This means that the patient is almost necessarily threatened by other consequent DM-damage caused to the eyes, the heart, the vessels, the kidneys, and to the gastro-intestinal tract. The early recognition of the diabetic foot or, expressed in other words, the proof of preserved health of the feet is of great interest to the physician and the patient.

An indicator plaster for diagnostics of the DF-syndrome is described in DE 100 18 790 A1. As indicator, the plaster contains cobalt-(II) chloride, which, in the dry state, has a blue coloration, and changes to a red coloration at a defined moisture content that is conforming to the normal moisture of the skin. If such a plaster is applied, for example to the sole of the foot, it is possible to determine after a short time whether or not a color change has occurred. In the known art, it is defined that the absence of a color change is indicative of an abnormal dryness of the skin that may constitute an indication of the diabetic foot.

A self-adhesive indicator is described in WO 97/31599, which permits indicating, for example the glucose content of the blood as a chemical reaction. The indicator therefore can be used for detecting the diabetes mellitus through a chemical reaction.

Finally, an indicator plaster for the diagnosis of diabetes is described in EP 04 30 608 A1. This plaster has an adhesive coating that is containing hydro-colloids that are soluble in water, and which has a band of liquid crystal that is sensitive to temperature and serves as the indicator. Like the other known plasters, this plaster has a relatively complicated technical structure and supplies its information as the result of thermal-chemical reactions.

The invention is based on the problem of providing aids or devices that are substantially acting mechanically or are activated for the early recognition of the diabetic foot for as many diabetics as possible who are still free of any symptoms of the diabetic foot. These aids are intended to be available at favorable cost and to be capable of supplying strong evidence (i.e. scientifically validated evidence), and to be uncomplicated in their application.

The solution as defined by the invention consists for the indicator plaster consists in that a pressure indicator that has to be activated, and which is therefore sensitive to pressure, is integrated in the body of the plaster and must be activated by the weight of the patient who is standing on the plaster. The indicator is preferably realized in the form of a pressure-proof ball. In addition to or as an alternative for the ball, it may be favorable if provision is made for a pressure indicator foil in the indicator that needs to be activated by the patient. A number of improvements and other embodiments of the invention are specified in the dependent claims.

If the pressure indicator as defined by the invention is realized in the form of a pressure-proof ball, the instruction that the indicator has to be activated by the weight of the patient who is standing on the plaster, means that the ball located in the indicator plaster on the sole of the foot, is perceived by the patient as a mechanical source of pain depending on whether the foot is healthy or not, or, in the extreme case, not perceived at all. As stated above, provision can be made also for a commercially available pressure indicator foil as the pressure indicator. Such a foil changes its color irreversibly at a defined pressure. Within the framework of the invention, the foil can be adapted in such a way that it indicates by a change in color that the critical pressure has been exceeded. The pressure values that are critical for causing the diabetic foot are known, and compiled in standard tables. It is particularly beneficial if the pressure indicator foil and the ball are combined in one and the same indicator plaster.

The pressure-proof ball is preferably located on the underside of the plaster that has to be facing the sole of the foot. When applied, the ball, which is preferably not visible, will then be located between the skin and the plaster. If necessary, provision has to be made for the indicator foil on the top side of the plaster, which has to be facing away from the sole of the foot, so that the effect of the pressure is not dampened by layers of the plaster. If the plaster is comprised of the ball and the pressure indicator foil, the ball is applied on the underside, and the pressure indicator foil is attached the top side of the plaster, preferably one on top of the other, so that the ball is capable of exerting pressure on the foil.

The plaster to be used as defined by the invention may preferably have an adhesive surface (on its underside), with the help of which it is fixed on the sole of the foot. However, an adhesive surface per se is not essential to the invention for securing the plaster. Other means for securing the ball and/or the indicator foil can be used as well, for example a dressing. Therefore, within the framework of the invention, the term “plaster” is generally understood to be a fixing means or also a carrier for the pressure indicator. Basically, the ball and/or the indicator foil can be fixed in the respective site of the skin with the help of an adhesive as well that is applied to the ball and/or the indicator foil. It may suffice the secure the components in such a manner that will allow the patient to walk a few steps with the plaster. According to the invention, a commercially available pressure indicator foil can be provided with the function of a plaster and can be directly equipped with one of the pressure-proof balls, and prepared for fixing it in the site to be examined in the given case.

Furthermore, the ball provided as defined by the invention, whereby it is possible to integrate two or more balls in a plaster as well, does not necessarily need to have the geometric shape of a sphere. It is also possible within the framework of the invention to make provision for oval or irregular shapes such as the shape of a small stone.

As in connection with a plaster, reference is simularly made herein to a ball only for the purpose of simplifying the description. Preferably, a commercially available plaster with an adhesive underside can be prepared in such away that when it is applied, the ball has to be fixed in a predetermined site of the skin between the support provided by the skin or wound, and the body of the plaster.

According to a further development of the invention, the ball integrated in the plaster has a diameter range in the order of magnitude of from 1.5 to 5 mm, and an approximate maximum diameter, i.e. a diameter adequately large that a plaster with a ball applied to his or her diseased foot (i.e. to the stepping surface of the sole of the foot) can no longer be safely distinguished from a plaster without such a ball, but that the difference is distinctly perceived by the healthy patient. The size of the ball can be selected in such a manner that it is individually adapted to the sensitivity and/or the body weight of the individual patent, for example by testing. In connection with the exemplified embodiments described above, a ball diameter of from about 2 to 4 mm has been found to be favorable, i.e. in the order of magnitude of 3 mm.

According to the invention, commercially available pressure indicator foils with the specified properties can be used, for example in the form of a separated double foil, or in the form of a double foil that has already been joined and supplied as one single part. Such foils are consisting of a color-supplying layer that is changing its color in response to a minimum pressure. Within the framework of the invention, the foil and/or the plaster, and if necessary the compressibility of the latter, are coordinated or adapted or selected in such a manner that the foil, in conjunction with the plaster, will provide a visible indication when a critical pressure is exceeded.

In the application of the invention with a pressure-proof ball (in conjunction with the pressure indicator foil, if need be), a plaster set is preferably provided that, in the delivered form, is comprised of a prepared plaster (with the ball) and an unprepared plaster. The patient is not told which one of the plasters has been prepared (blind test). A plaster is fixed on each sole of the feet of the patient. The patient is then made to walk a few steps and asked about the sensation he or she felt. Preferably, the two plasters are fixed in the same or corresponding sites of the right and the left foot, and specifically on the actual stepping surfaces of the soles of the feet. In some cases, it may be favorable if the test is repeated with a second set of plasters, however, with a reversed distribution.

The sensation to be expected from a patient on account of the ball on his or her healthy foot consists of an emphatic to painful feeling in the site of application of the plaster that has been prepared with a ball. The ball is substantially acting in said site like a stone in a shoe. If this sensation is missing or incorrectly localized, this is a safe indication that a diabetic foot is in its early stage. It may be beneficial in this sense if the ball is integrated in the plaster near the underside of the latter, and preferably directly on the underside of the body of the plaster, because in this way, the effect of the ball is not dampened by intermediate layers, i.e. layers disposed between the skin and the ball.

In connection with the application of the indicator foil as well, it may be favorable if a plaster with the indicator foil is applied to the actual stepping surfaces of each of the two feet of the patient. Differences between the feet can be diagnosed in this way. This is favorable mainly in the DF-examination because with diabetes mellitus, only one foot is diseased (first) in most cases.

As stated above, it may be beneficial within the framework of the invention if the pressure indicator foil is combined with a pressure-proof ball, or vice versa. A ball is referred to as a “pressure proof” ball because the pressure indicator foils may contain dye balls that are crushed when they are subjected to a minimum pressure, i.e., such balls are not pressure-proof. Since the pressure-proof balls may exert effects on the skin without, however, altering the indicator foil itself, it may be favorable if provision is made for the ball on the underside of the plaster, and for the indicator foil on the top side of the plaster. In that case, the effect of the ball will not be dampened by intermediate layers, i.e. this effect will be maximal and any change of the foil disposed externally can be easily detected.

Also, if the invention is applied with the pressure indicator foil, a plaster set is preferably provided that is comprised of two prepared plasters (i.e. prepared with the pressure indicator foil). A plaster is applied to each foot sole of the patient. The patent is then asked to walk a few steps and the pressure indicator foil is then examined to determine whether a change in color or the like has occurred. If the pressure indicator foil is provided in connection with the aforementioned set of plasters on both plasters, and the ball is provided on one of the plasters, the protective sensitivity (e.g. the surface sensitivity and the deep sensitivity, as well as the sensation of pain) and the critical pressure on the foot sole can be tested at the same time.

The invention permits for the first time the detection of the crucial risk parameters for the DF in a mechanical and uncomplicated manner and at favorable cost. The indicator plaster as defined by the invention is economically beneficial and scientifically founded; it incurs low costs and supplies a conclusive statement for a healthy or a diseased foot. The patient can conduct the test on his or her own also without the physician.

Details of the invention are explained with the help of the schematic representation of an exemplified embodiment shown in the drawing, in which:

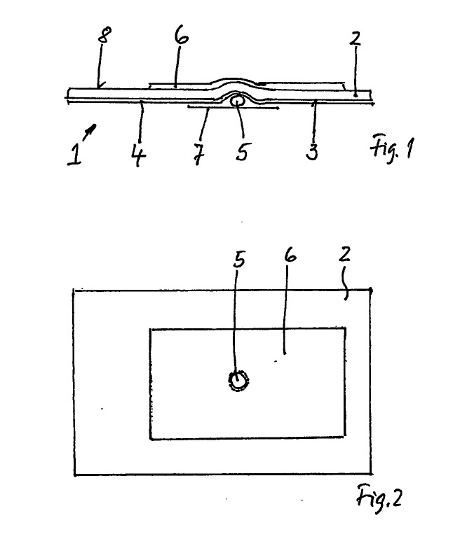

FIG. 1 is a section through an indicator plaster as defined by the invention; and

FIG. 2 is a top view of the indicator plaster according to FIG. 1.

The indicator plaster according to FIGS. 1 and 2, which is denoted by 1 as a whole, is substantially comprised of a support layer 2 with the adhesive layer 4 provided on the underside 3 of the support layer. A pressure-proof ball 5 and a pressure indicator foil 6 are integrated in the indicator plaster 1 according to FIG. 1. According to FIG. 1, the ball 5 is located on the underside of the support layer 2, and preferably at the same time on the underside of the adhesive layer 4, where it may be covered by a thin sheet 7. According to FIG. 1, the pressure indicator foil 6 is located on the top side 8 of the support layer 2, said top side opposing the underside 3. The ball 5 and the pressure indicator foil are preferably disposed one on top of the other, so that the ball 5 is directly acting on the foil 6 when pressure is exerted.

1. A medical indicator plaster (1) for application to the sole of the foot, for use in the early detection of the diabetic foot (DF), characterized in that at least one pressure indicator (5, 6) to be activated by the weight of the patient standing on the plaster, is integrated in the body of the plaster.

2. The indicator plaster according to claim 1, characterized in that the pressure indicator is realized in the form of a pressure-proof ball (5).

3. The indicator plaster according to claim 1, characterized in that the pressure indicator is realized in the form of a pressure indicator foil.

4. The indicator plaster according to any one of claims 1 to 3, characterized in that provision is made at the same time for at least one pressure-proof ball (5) and at least one pressure indicator foil (6) preferably disposed one on top of the other.

5. The indicator plaster according to any one of claims 1 to 4, characterized in that the pressure-proof ball (5) is mounted on the underside (3) of the plaster (1) facing the sole of the foot in the application.

6. The indicator plaster according to at least one of claims 1 to 5, characterized in that the diameter of the pressure-proof ball is particularly in the order of magnitude of from 1.5 to 5 mm, preferably in the order of about from 2 to 4 mm, and is selected in such a manner that a plaster with the ball (5) fixed in the application under the sole of the foot cannot be safely distinguished from a plaster without the ball by a patient with a diabetic foot.

7. The indicator plaster according to at least one of claims 1 to 6, characterized by a form of delivery in the form of a set comprised of one plaster (1) with the ball (5), and one plaster without the ball.

8. The indicator plaster according to at least one of claims 1 to 7, characterized in that a pressure indicator foil (6) integrated in the plaster (1) is applied to the top side (8) of the plaster (1), said top side having to face away from the sole of the foot in the application.

9. The use of a plaster (1) to be applied to the sole of the foot of a patient and equipped with a pressure indicator, in particular in the form of a pressure-proof ball (5) and/or a pressure indicator foil (6), in the medical diagnosis preferably for the early detection of the diabetic foot.
